(19) United States
(12) Patent Application Publication
(10) Pub. No.: US 2024/0090746 A1
(43) Pub. Date: Mar. 21, 2024

(54) INSERTION DEVICE

(71) Applicant: OLYMPUS MEDICAL SYSTEMS CORP., Tokyo (JP)

(72) Inventors: Nagahide SAKAI, Tokyo (JP); Satoshi HORIE, Tokyo (JP); Eijiro SATO, Tokyo (JP)

(73) Assignee: OLYMPUS MEDICAL SYSTEMS CORP., Tokyo (JP)

(21) Appl. No.: 18/211,700

(22) Filed: Jun. 20, 2023

Related U.S. Application Data
(60) Provisional application No. 63/356,055, filed on Jun. 28, 2022.

(51) Int. Cl. A61B 1/00; A61B 1/015; A61B 1/05

(52) U.S. Cl. CPC A61B 1/00096; A61B 1/00087; A61B 1/00094; A61B 1/015; A61B 1/05

(57) ABSTRACT

Insertion equipment includes an insertion section, a distal end member provided at a distal end of the insertion section, and a tube configured to supply a fluid to the distal end member. In the distal end member, the fluid passes through a first flow channel extending in a distal end direction, passes through a second flow channel in a direction different from the distal end direction, is divided, is discharged through a first opening in a distal end surface, and part of the fluid is discharged through one or more second openings in an outer circumferential surface of the distal end member in a radial direction or a proximal end direction.

### CROSS REFERENCE TO RELATED APPLICATION

This application claims benefit of Provisional Application No. 63/356,055 filed in United States of America on Jun. 28, 2022, the entire contents of which are incorporated herein by this reference.

### BACKGROUND OF THE INVENTION

### 1. Field of the Invention

The present invention relates to insertion equipment including flow channels through which a fluid can be supplied to the inside of a subject.

### 2. Description of Related Art

Insertion equipment such as an endoscope is widely used in the medical field. The insertion equipment has an elongated insertion section that can be inserted into a subject. A user inserts the insertion section into the subject to perform observation of the inside of the subject, treatment for a lesion area in the subject, and the like.

The insertion equipment such as an endoscope commonly has a conduit for guiding a fluid such as a liquid to a distal end side of the insertion section. The fluid guided by the conduit is squirted through an opening of a nozzle or the like provided for a distal end member (Japanese Patent Application Laid-Open Publication No. 2010-46300). The fluid guided by the conduit to the distal end member is used for cleaning of an observation window, and the like.

In insertion equipment such as an ureteroscope, a fluid guided by a conduit is perfused into a subject. The fluid accumulated in the subject by the perfusion dilates the inside of the subject. A visual field in the subject is enlarged accordingly. The fluid ejected by perfusion to the outside of the subject conveys broken calculi or the like.

### SUMMARY OF THE INVENTION

Insertion equipment of an embodiment of the present invention includes: an insertion section inserted into a subject; a distal end member provided at a distal end of the insertion section; and a tube configured to supply a fluid to the distal end member. In the distal end member, the fluid passes through a first flow channel extending in a distal end direction, passes through a second flow channel in a direction different from the distal end direction, is divided, is discharged through a first opening in a distal end surface, and part of the fluid is discharged through one or more second openings in an outer circumferential surface of the distal end member in a radial direction or a proximal end direction.

Insertion equipment of another embodiment of the present invention includes: an insertion section inserted into a subject; a distal end member provided at a distal end of the insertion section; at least one sensor disposed in the distal end member and configured to detect a physical quantity; and a tube configured to supply a fluid to the distal end member. In the distal end member, the fluid passes through a first flow channel extending in a distal end direction, passes through a second flow channel in a direction different from the distal end direction, is divided, is discharged through a first opening in a distal end surface, and part of the fluid passes through the second flow channel and is discharged through one or more second openings in an outer circumferential surface of the distal end member. A first angle formed by a first line that connects a central axis of the distal end member and the second openings and a second line that connects the central axis and the sensor is more than 10 degrees.

### BRIEF DESCRIPTION OF THE DRAWINGS

FIG. 1 is a perspective view showing an appearance of an endoscope of a first embodiment;

FIG. 2 is a perspective view showing a distal end member of the first embodiment from a distal end side;

FIG. 3 is a plan view of the distal end member of the first embodiment;

FIG. 4 is a Iv-Iv cross-sectional view of FIG. 3 of the first embodiment;

FIG. 5 is an exploded perspective view of the distal end member of the first embodiment;

FIG. 6 is a perspective view of a first distal end member of the first embodiment from a proximal end side;

FIG. 7 is an explanatory diagram showing a behavior of a fluid in the distal end member of the first embodiment;

FIG. 8A is a schematic diagram of FIG. 7 showing the behavior of the fluid in the distal end member;

FIG. 8B is a schematic diagram showing the behavior of the fluid in the distal end member;

FIG. 9 is a schematic diagram showing a behavior of a fluid in a distal end member of Modification 1 of the first embodiment;

FIG. 10 is a schematic diagram showing a behavior of a fluid in a distal end member of Modification 2 of the first embodiment;

FIG. 11 is a schematic diagram showing a behavior of a fluid in a distal end member of Modification 3 of the first embodiment;

FIG. 12 is a schematic diagram showing a behavior of a fluid in a distal end member of Modification 4 of the first embodiment;

FIG. 13 is a schematic diagram showing a behavior of a fluid in a distal end member of Modification 5 of the first embodiment;

FIG. 14 is a schematic diagram showing a behavior of a fluid in a distal end member of Modification 6 of the first embodiment;

FIG. 15 is a schematic diagram showing a behavior of a fluid in a distal end member of Modification 7 of the first embodiment;

FIG. 16 is a schematic diagram showing a behavior of a fluid in a distal end member of Modification 8 of the first embodiment;

FIG. 17 is a schematic diagram showing a behavior of a fluid in a distal end member of Modification 9 of the first embodiment;

FIG. 18 is a schematic diagram showing a behavior of a fluid in a distal end member of Modification 10 of the first embodiment;

FIG. 19 is a schematic diagram showing a behavior of a fluid in a distal end member of Modification 11 of the first embodiment;

FIG. 20 is a schematic diagram showing a behavior of a fluid in a distal end member of Modification 12 of the first embodiment;

FIG. 21 is a perspective view of a distal end member of a second embodiment;

FIG. 22A is a side view of the distal end member of the second embodiment;

FIG. 22B is a front view showing a position of a sensor in the distal end member of the second embodiment;

FIG. 23 is a side view showing the position of the sensor in the distal end member of the second embodiment;

FIG. 24 is a partial perspective view showing an arrangement state of the sensor in the distal end member of the second embodiment;

FIG. 25A is a plan view of the sensor of an endoscope of the second embodiment;

FIG. 25B is a plan view of the sensor of the endoscope of the second embodiment;

FIG. 25C is a plan view of the sensor of the endoscope of the second embodiment;

FIG. 25D is a plan view of the sensor of the endoscope of the second embodiment;

FIG. 26A is a plan view of a composite sensor of the endoscope of the second embodiment;

FIG. 26B is a cross-sectional view taken along the line XXIVB-XXVIB in FIG. 26A;

FIG. 26C is a cross-sectional view of the composite sensor of the endoscope of the second embodiment;

FIG. 27A is a plan view of a composite sensor of the endoscope of the second embodiment;

FIG. 27B is a cross-sectional view taken along the line XXVIIB-XXVIIB in FIG. 27A;

FIG. 27C is a cross-sectional view of a composite sensor of the endoscope of the second embodiment;

FIG. 28A is a cross-sectional view of a composite sensor of the endoscope of the second embodiment;

FIG. 28B is a cross-sectional view of a composite sensor of the endoscope of the second embodiment;

FIG. 28C is a cross-sectional view of a composite sensor of the endoscope of the second embodiment;

FIG. 29A is a side view of a composite sensor of the endoscope of the second embodiment;

FIG. 29B is a side view of a composite sensor of the endoscope of the second embodiment;

FIG. 29C is a side view of a composite sensor of the endoscope of the second embodiment;

FIG. 30A is a perspective view of a distal end member of Modification 1 of the second embodiment;

FIG. 30B is a cross-sectional view of the distal end member of Modification 1 of the second embodiment;

FIG. 31A is a perspective view of a distal end member of Modification 2 of the second embodiment;

FIG. 31B is a cross-sectional view of the distal end member of Modification 2 of the second embodiment;

FIG. 32A is a perspective view of a distal end member of Modification 3 of the second embodiment;

FIG. 32B is a cross-sectional view of the distal end member of Modification 3 of the second embodiment;

FIG. 33A is a perspective view of a distal end member of Modification 4 of the second embodiment; and

FIG. 33B is a cross-sectional view of the distal end member of Modification 4 of the second embodiment.

### DETAILED DESCRIPTION OF PREFERRED EMBODIMENTS

To perform perfusion in insertion equipment such as an endoscope, a flow quantity of a fluid needs to be ensured. Along with recent reduction in diameter of an insertion section of the insertion equipment, a conduit is reduced in diameter. In insertion equipment having a small-diameter conduit, a flow velocity of a fluid flowing in the conduit increases when a flow quantity of the fluid is ensured. A flow velocity distribution occurs in the fluid flowing in the conduit. In other words, the velocity of the fluid flowing at the center of the conduit is faster than the velocity of the fluid flowing in the vicinity of a wall surface of the conduit. In a case where the fluid is discharged from the small-diameter conduit directly into the subject, the fast flow might cause an observation target and a treatment target to be considerably moved.

Thus, it is desirable that the fluid perfused into the subject should be discharged through an opening in a distal end member at such a slow flow velocity that the observation target and the treatment target are not moved.

Embodiments which will be described below can provide insertion equipment that reduces the flow velocity distribution of a discharged fluid while ensuring a flow quantity of the fluid when performing perfusion.

### First Embodiment

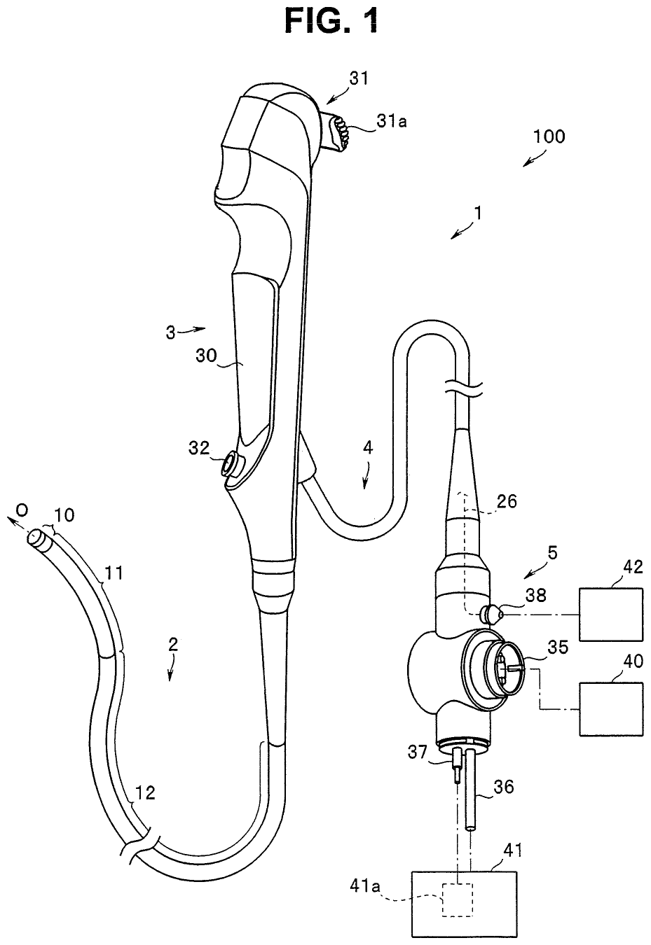

Hereinafter, a first embodiment of the present invention will be described with reference to FIG. 1 to FIG. 8. Note that in the present embodiment, an endoscope 1 will be described as an example of insertion equipment.

### <Configuration of Endoscope>

The endoscope 1 shown in FIG. 1 is an ureteroscope. The endoscope 1 is a single-use endoscope to be discarded (disposed of) after a single use. The endoscope of the present invention may be a reuse endoscope that is disinfected and sterilized after use to be reutilized. The endoscope may be for industrial use.

The endoscope 1 has an insertion section 2, an operation section 3, a universal cable 4, and an endoscope connector 5. The insertion section 2 has a distal end member 10, a bending portion 11, and a flexible tube portion 12 sequentially from the distal end side.

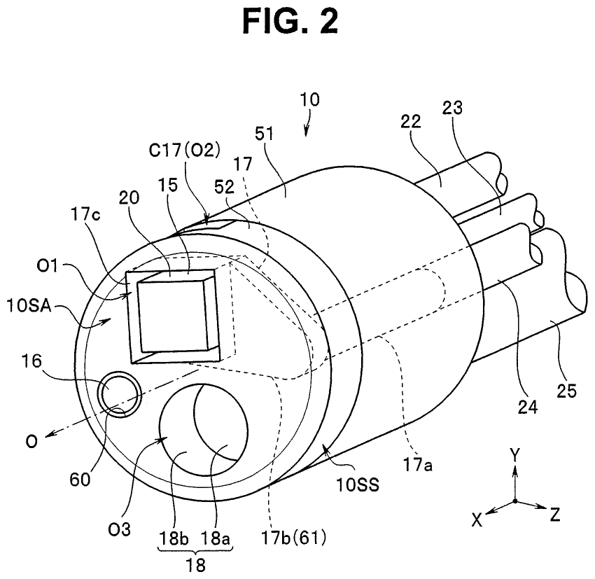

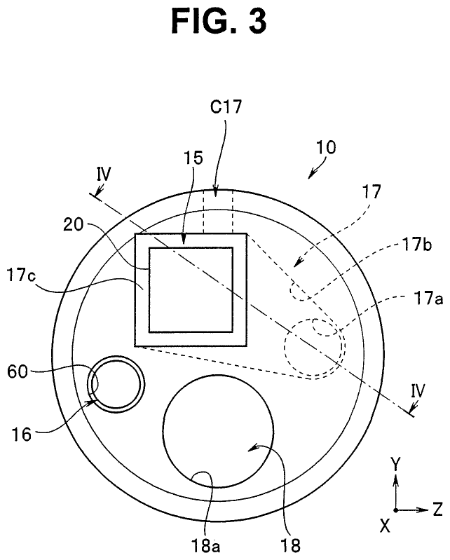

As shown in FIG. 2 and FIG. 3, the distal end member 10 is provided with an image pickup unit 15, an illumination unit 16, a gas/liquid feeding flow channel 17, and a treatment instrument hole 18. Hereinafter, a distal end direction O of the insertion section 2 shall be an X-axis value increasing direction in a triaxial orthogonal coordinate system.

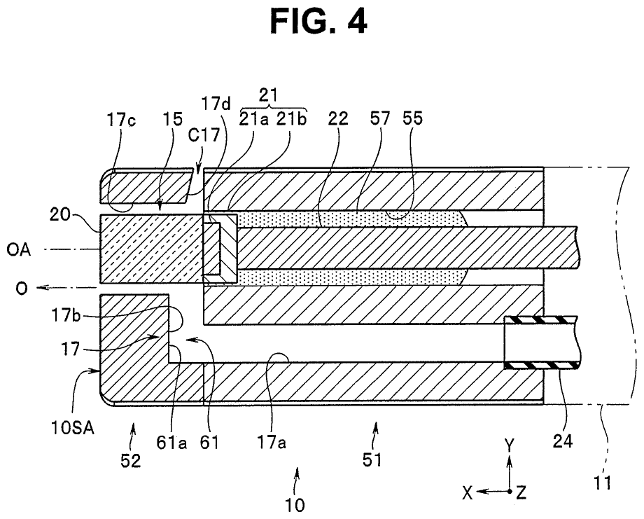

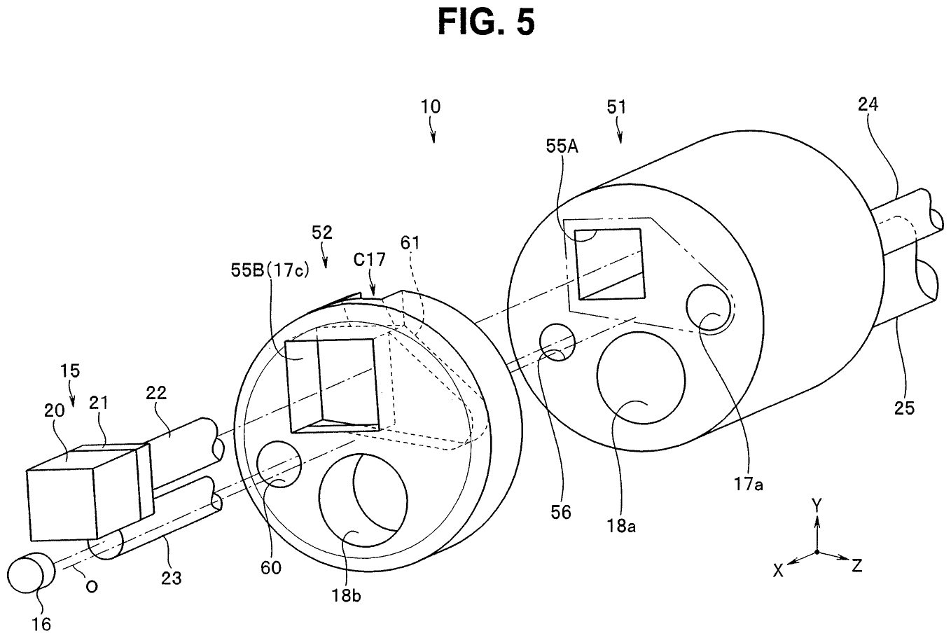

As shown in FIG. 4 and FIG. 5, the image pickup unit 15 includes a lens unit 20 and an image pickup device unit 21. The lens unit 20 is configured by a laminate of a plurality of objective lenses. A plan view shape of the lens unit 20 forms a rectangular shape. The image pickup device unit 21 has an image pickup device 21a and a circuit board 21b. The image pickup device unit 21 is fixed to the lens unit 20. An image of a subject is formed on the image pickup device 21a by the lens unit 20. The image pickup device 21a receives an optical image focused by the lens unit 20 for conversion into an electric signal.

The image pickup device 21a is mounted on the circuit board 21b. A plurality of signal cables are connected to the circuit board 21b. The plurality of signal cables are bundled to configure a signal cable bundle 22. A plan view shape of the circuit board 21b forms a rectangular shape having a size substantially identical to the plan view shape of the lens unit 20. An overall shape of the image pickup unit 15 forms a substantially prismatic shape accordingly.

The bending portion 11 has a configuration that can actively bend the insertion section 2 in upward and downward two directions (UP-DOWN). The flexible tube portion 12 is configured by a tubular member that is passively deflected by an external force. The inside of the flexible tube portion 12 allows insertion of the signal cable bundle 22, a light guide bundle 23, a gas/liquid feeding channel 24, a treatment instrument channel 25, and the like.

The illumination unit 16 is optically connected to a distal end side of the light guide bundle 23 that guides illumination light.

The gas/liquid feeding channel 24 is a tube for supplying a fluid (gas or liquid). A distal end side of the gas/liquid feeding channel 24 is connected to a proximal end side of the gas/liquid feeding flow channel 17 of the distal end member 10.

The treatment instrument channel 25 is a tube for guiding a treatment instrument or the like. A distal end side of the treatment instrument channel 25 is connected to a proximal end side of the treatment instrument hole 18 of the distal end member 10. The treatment instrument inserted into the treatment instrument channel 25 is used in a state of being partially discharged through an opening (a third opening O3) in a distal end surface 10SA of the treatment instrument hole 18. Note that in the endoscope 1, the treatment instrument channel 25 also has a function as an ejection tube that suctions a fluid and the like in the subject as will be described later.

The operation section 3 is provided with a grasping portion 30, a bending operation portion 31, and a pipe sleeve 32. The bending operation portion 31 is provided with a bending operation lever 31a. A user performs a rotation operation of the bending operation lever 31a to perform a bending operation of the bending portion 11.

The pipe sleeve 32 is provided on the distal end side relative to the grasping portion 30. A proximal end side of the treatment instrument channel 25 is connected to the pipe sleeve 32. In other words, the pipe sleeve 32 is communicatively connected to the treatment instrument hole 18 by way of the treatment instrument channel 25.

The universal cable 4 allows insertion of the signal cable bundle 22, the light guide bundle 23, the gas/liquid feeding channel 24, a suction channel 26 (see FIG. 1), and the like.

A distal end side of the suction channel 26 is connected to the treatment instrument channel 25. In other words, the suction channel 26 is communicatively connected to the treatment instrument hole 18 by way of the treatment instrument channel 25. Thus, the treatment instrument channel 25 and the suction channel 26 have a function as an ejection tube that sends a liquid in the subject to the outside of the body.

The endoscope connector 5 is connected to an extended end of the universal cable 4. The endoscope connector 5 has an electric connector 35, a light source connector 36, a gas/liquid feeding plug 37, and a suction pipe sleeve 38.

The signal cable bundle 22 is connected to the electric connector 35. The electric connector 35 is connected to a video processor 40 (see FIG. 1). The light guide bundle 23 is connected to the light source connector 36. The light source connector 36 is connected to a light source device 41 (see FIG. 1).

The gas/liquid feeding channel 24 is connected to the gas/liquid feeding plug 37. The gas/liquid feeding plug 37 is connected to a gas/liquid feeding device 41a. In the present embodiment, the gas/liquid feeding device 41a is a metering pump. A suction tube is connected to the suction pipe sleeve 38. The suction pipe sleeve 38 is connected to a suction device 42 (see FIG. 1).

When the endoscope connector 5 is connected to external equipment such as the gas/liquid feeding device 41a, the endoscope 1 configures an endoscope system 100 that perfuses a fluid between the inside and outside of the subject. Note that the endoscope connector 5 may be connected to the external equipment using an adapter which is an intermediate connection member not shown.

### <Configuration of Distal End Member>

The distal end member 10 has a first distal end member 51 and a second distal end member 52. A proximal end side of the first distal end member 51 having a substantially cylindrical shape is connected to the bending portion 11.

As shown in FIG. 5, the first distal end member 51 is provided with an image pickup unit hole 55A, a light guide hole 56, a first flow channel 17a, and a first treatment instrument hole 18a.

The image pickup unit hole 55A is configured by a rectangular hole extending through the first distal end member 51. As shown in FIG. 4, the image pickup device unit 21 and the signal cable bundle 22 are inserted in the image pickup unit hole 55A. The image pickup device unit 21 and the signal cable bundle 22 are fixed to the inside of the image pickup unit hole 55A with an adhesive agent 57.

The light guide hole 56 is configured by a circular hole extending through the first distal end member 51. The light guide bundle 23 inserted in the light guide hole 56 is fixed to the first distal end member 51 with an adhesive agent or the like.

The first flow channel 17a is configured by a wall of a circular hole extending through the first distal end member 51. The first flow channel 17a is an upstream portion of the gas/liquid feeding flow channel 17 (at the time of perfusion, a liquid feeding flow channel). As shown in FIG. 4, a distal end portion of the gas/liquid feeding channel 24 is connected to a proximal end portion of the first flow channel 17a. The inner diameter of the first flow channel 17a is substantially the same diameter as the inner diameter of the gas/liquid feeding channel 24.

The first treatment instrument hole 18a is configured by a wall of a circular hole extending through the first distal end member 51. A distal end portion of the treatment instrument channel 25 is connected to a proximal end portion of the first treatment instrument hole 18a.

The outer diameter of the second distal end member 52 having a substantially cylindrical shape is substantially the same diameter as the outer diameter of the first distal end member 51. A proximal end surface of the second distal end member 52 is fixed to a distal end surface of the first distal end member 51 by adhesion or the like.

As shown in FIG. 3 to FIG. 6, the second distal end member 52 is provided with an illumination unit hole 60, a guide groove 61, a second image pickup unit hole 55B (hereinafter also referred to as an “image pickup unit hole 55B”), a second treatment instrument hole 18b, and a cutout C17.

The illumination unit hole 60 is configured by a circular hole extending through the second distal end member 52. The illumination unit hole 60 is arranged at a position opposite to the light guide hole 56.

The illumination unit 16 is inserted in a distal end side of the illumination unit hole 60. The illumination unit 16 is fixed to the second distal end member 52 using an adhesive agent or the like.

A distal end portion of the light guide bundle 23 is inserted in a proximal end side of the illumination unit hole 60. The illumination unit 16 is optically connected to the light guide bundle 23 accordingly.

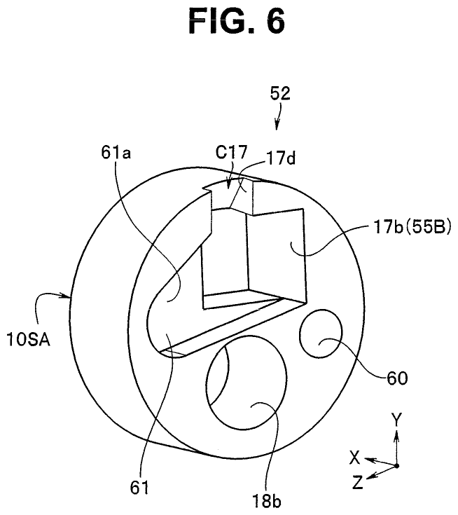

As shown in FIG. 6, the guide groove 61 is provided in the proximal end surface of the second distal end member 52. The guide groove 61 is configured by a recessed wall having a predetermined depth. The guide groove 61 extends from the first flow channel 17a side to the image pickup unit hole 55B side as shown by a broken line in FIG. 5.

The cutout C17 is provided in the proximal end surface of the second distal end member 52. The cutout C17 communicates with the guide groove 61.

The second treatment instrument hole 18b is configured by a circular hole extending through the second distal end member 52. The second treatment instrument hole 18b configures the treatment instrument hole 18 in conjunction with the first treatment instrument hole 18a.

The endoscope 1 performs perfusion for dilating the inside of a body cavity (a lumen or an organ in the subject) to ensure a visual field and for ejecting a solid material piece or the like in the body cavity (lumen) to the outside of the body. At the time of perfusion, a fluid (liquid) such as normal saline, for example, is supplied to the distal end member 10 by way of the gas/liquid feeding channel 24. The fluid supplied to the distal end member 10 is discharged to the inside of the body by way of the gas/liquid feeding flow channel 17. Part of the fluid discharged to the inside of the body is suctioned through the treatment instrument hole 18. The fluid suctioned through the treatment instrument hole 18 is ejected to the outside of the body by way of the treatment instrument channel 25 and the suction channel.

At the time of perfusion, the fluid supplied from the small-diameter gas/liquid feeding channel 24 to the distal end member 10 is discharged to the inside of the body after the flow velocity is averaged by the gas/liquid feeding flow channel 17, as will be described later. The fluid discharged through the gas/liquid feeding flow channel 17 cleans the lens unit 20.

### <Configuration of Flow Channel>

As shown in FIG. 4, the gas/liquid feeding flow channel 17 has the first flow channel 17a, a second flow channel 17b, a third flow channel 17c, and a fourth flow channel 17d.

When the second distal end member 52 is fixed to the first distal end member 51, a wall surface of the guide groove 61 forms a wall of the second flow channel 17b in conjunction with the distal end surface of the first distal end member 51. An upstream side of the second flow channel 17b is connected to a downstream end of the first flow channel 17a. The second flow channel 17b configures an intermediate flow portion of the gas/liquid feeding flow channel 17. The cutout C17 forms a wall of the fourth flow channel 17d in conjunction with the distal end surface of the first distal end member 51.

The second flow channel 17b is a flow channel that sends the fluid having passed through the first flow channel 17a extending in the distal end direction O in a direction different from the distal end direction O. A wall 61a which is part of the wall surface of the guide groove 61 is opposite to the first flow channel 17a. As will be described later, the wall 61a is a flow buffer wall with which the fluid collides.

The second flow channel 17b circulates the fluid having passed through the first flow channel 17a in a direction (the radial direction: a Z-axis direction) orthogonal to the distal end direction O. A downstream side of the second flow channel 17b is set toward the image pickup unit 15 arranged in the image pickup unit hole 55B.

In the present embodiment, a groove width of the guide groove 61 gradually increases from the first flow channel 17a side toward the image pickup unit hole 55B side. A flow channel cross-sectional area of the second flow channel 17b is larger than the flow channel cross-sectional area of the first flow channel 17a in the whole region from the upstream side to downstream of the second flow channel 17b.

The third flow channel 17c is a downstream portion of the gas/liquid feeding flow channel 17. An opening area of the image pickup unit hole 55B (an opening area of a plane (a YZ plane) orthogonal to the distal end direction O) is larger than the cross-sectional area (the area of the YZ plane) of the lens unit 20. The third flow channel 17c having a frame shape is configured by an inner wall of the image pickup unit hole 55B and an outer wall of the lens unit 20. When observed from the distal end side, the third flow channel 17c having a frame shape extends in the distal end direction O of the insertion section 2.

An opening having a frame shape of the distal end surface 10SA is a first opening O1, and an opening of the cutout C17 in an outer circumferential surface loss is a second opening O2. The first opening O1 is a discharge port of a main flow channel, and the second opening O2 is an opening of a sub-flow channel.

### <Flow of Fluid>

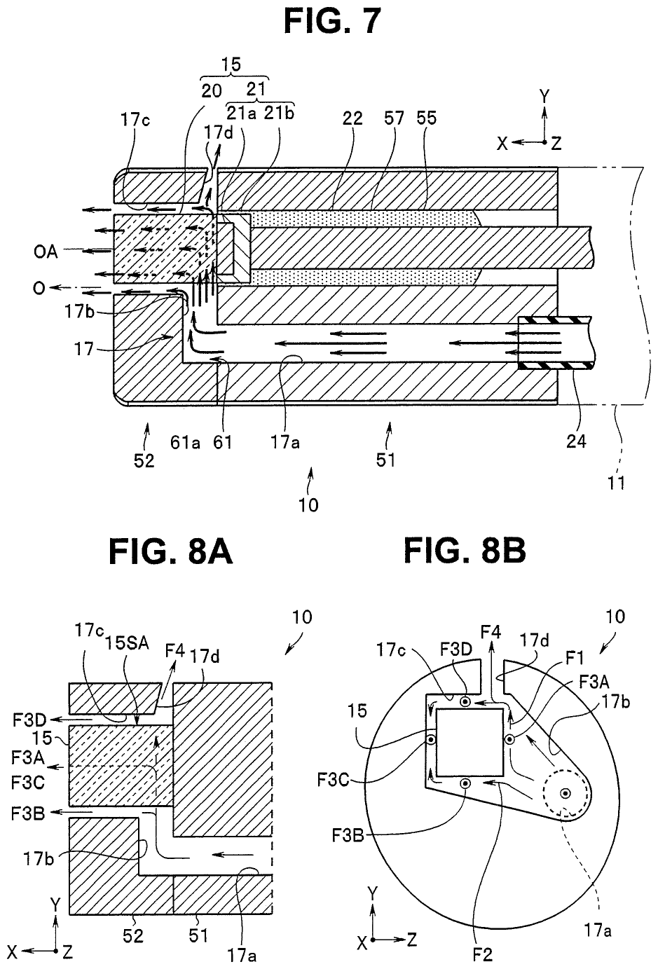

FIG. 8A is a schematic view of FIG. 7 showing a flow of a fluid. FIG. 8B is a schematic diagram showing the flow of the fluid at the cross section (the YZ plane) orthogonal to an insertion direction of the distal end member 10. In the drawing, circles surrounding black circles show the flow of the fluid in a near side direction on the sheet of drawing.

As shown in FIG. 7, FIG. 8A, and FIG. 8B, the first flow channel 17a extends in the distal end direction O similarly to the gas/liquid feeding channel 24. The cross-sectional area of the first flow channel 17a is substantially identical to the cross-sectional area of the gas/liquid feeding channel 24. Consequently, the fluid flowing from the gas/liquid feeding channel 24 to the first flow channel 17a is not decelerated, and flows through the first flow channel 17a as a laminar flow having a flow velocity distribution substantially the same as the flow velocity distribution of the gas/liquid feeding channel 24.

The fluid having passed through the first flow channel 17a flows in the second flow channel 17b. The fluid having flown in the second flow channel 17b collides with the wall 61a. With the collision with the wall 61a, the fluid is stirred to be turbulent, so that the flow velocity distribution of the fluid decreases.

The fluid after collision with the wall 61a flows to the downstream side of the second flow channel 17b along the wall 61a. An extending direction of the second flow channel 17b is different from an extending direction of the first flow channel 17a. Thus, the fluid is further stirred inside the second flow channel 17b, so that the flow velocity distribution decreases.

In the present embodiment, a width (a dimension on the YZ plane) of the second flow channel 17b expands toward the downstream side. Consequently, the fluid is reduced in flow velocity and diffused toward the downstream side of the second flow channel 17b. Note that as the depth of the guide groove 61 is deeper, the cross-sectional area of the second flow channel 17b increases, so that the fluid is reduced in flow velocity and diffused. Further, when the depth of the guide groove 61 is deeper, the distance from the first flow channel 17a of the first distal end member 51 to the wall 61a is longer, so that diffusion of the fluid is promoted.

In the second flow channel 17b, the fluid is divided by the lens unit 20 into three directions. In other words, as shown in FIG. 8A and FIG. 8B, the fluid is divided into a first flow F1, a second flow F2, and a third flow F3A. The first flow F1 flows in an upward direction (a Y-axis value increasing direction) on the sheet of drawing. The second flow F2 flows in a left direction (a Z-axis value decreasing direction) on the sheet of drawing. The third flow F3A flows in the third flow channel 17c in the near side direction (the X-axis value increasing direction) on the sheet of drawing along four side surfaces 15SA of the image pickup unit 15. The lens unit 20 which is a substantially quadrangular prism has a function of branching the flow of the fluid.

The first flow F1 and the second flow F2 are divided into third flows F3B, F3C, and F3D in the third flow channel 17c flowing in the near side direction (the X-axis value increasing direction) on the sheet of drawing while flowing around the lens unit 20. Part of the first flow F1 is discharged to the inside of the body by way of the fourth flow channel 17d of the cutout C17.

The third flow channel 17c is different from the extending direction of the second flow channel 17b. The difference in extending direction forcibly changes the direction in which the fluid flows, so that the fluid is further stirred.

A flow channel cross-sectional area of the third flow channel 17c is larger than the flow channel cross-sectional area of the first flow channel 17a. Further, part of the fluid is also discharged through the fourth flow channel 17d.

Thus, the fluid having passed through the third flow channel 17c is discharged from the distal end surface 10SA to the inside of the subject at a slow flow velocity with a small flow velocity distribution. The fluid having passed through the fourth flow channel 17d is discharged in the proximal end direction which is an opposite direction of a visual field direction of the lens unit 20. An inner space of the subject is dilated by the discharged fluid. This ensures a visual field in the inside of the subject. The fluid having passed through the fourth flow channel 17d may be discharged in the radial direction of the cylindrical distal end member 10. However, the fluid discharged in the proximal end direction can prevent the observation target, the treatment target, and the like from being moved by the fluid as compared with the fluid discharged in the radial direction.

The lens unit 20 is cleaned by the fluid discharged through the third flow channel 17c. Air bubbles, broken calculi, and the like adhering to the lens unit 20 are removed by the fluid discharged through the third flow channel 17c. This ensures a field of view of the lens unit 20.

The fluid accumulated in the inside of the subject is suctioned through the treatment instrument hole 18. The fluid suctioned through the treatment instrument hole 18 is ejected to the outside of the body by way of the treatment instrument channel 25 and the suction channel. These achieve perfusion of the fluid in the inside of the subject.

The first flow channel 17a to the third flow channel 17c that configure the gas/liquid feeding flow channel 17 are connected in a crank shape. Thus, the gas/liquid feeding flow channel 17 can reduce the flow velocity distribution of the fluid and decelerate the flow velocity in the fastest flow velocity region by stirring the fluid of a laminar flow having a large flow velocity distribution supplied from the gas/liquid feeding channel 24 to be turbulent. Part of the fluid supplied from the gas/liquid feeding channel 24 is also discharged through the fourth flow channel 17d.

Even if a fluid including a flow having a fast flow velocity is supplied from the small-diameter gas/liquid feeding channel 24, the distal end member 10 prevents the fluid from being intensely discharged from the distal end to the inside of the subject. The distal end member 10 can prevent the observation target, the treatment target, and the like from being moved by the fluid while ensuring the flow quantity of the fluid to be supplied to the inside of the subject.

In a case of laser-fracturing a kidney calculus, the endoscope 1 can cool the inside of calices and improve the visual field while preventing the kidney calculus to be laser-fractured from scattering. The endoscope 1 can achieve efficient laser fracturing of the kidney calculus.

The cutout C17 is formed in the surface on the proximal end side of the second distal end member 52. Thus, the fourth flow channel 17d is easily formed by attaching the first distal end member 51 and the second distal end member 52. Note that the fourth flow channel 17d may be a through-hole having an opening in the outer circumferential surface 10SS of the second distal end member 52 and communicating with the second flow channel 17b. The fourth flow channel 17d may be a cutout in the surface of the first distal end member 51 on the distal end side.

### <Modifications of First Embodiment>

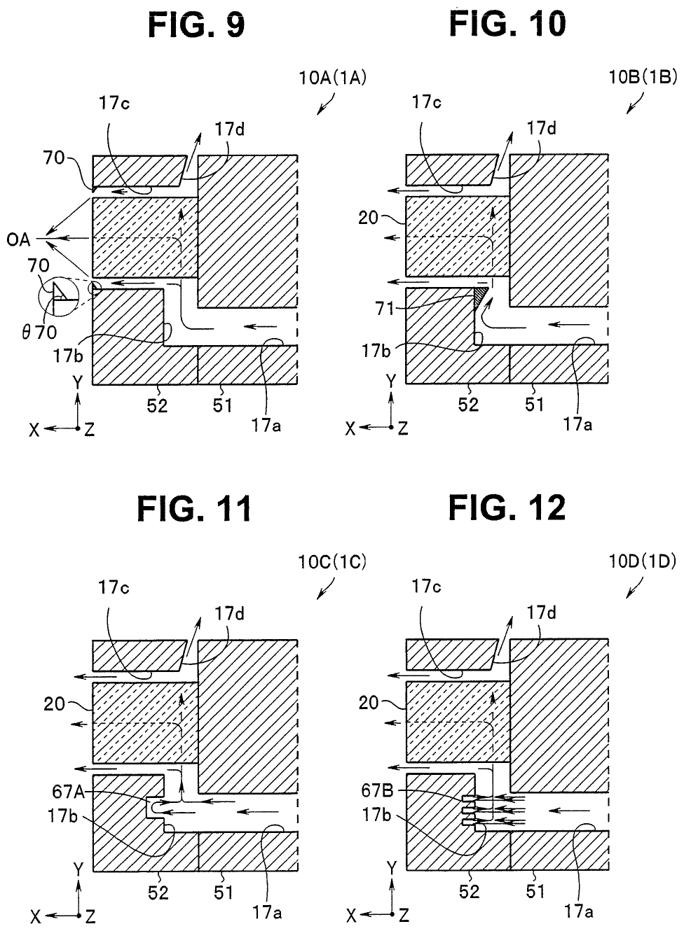

Modifications 1 to 12 of the first embodiment will be described with reference to FIG. 9 to FIG. 20. Modifications 1 to 12 of the first embodiment are similar to the first embodiment and have the same effects as the effects of the first embodiment. Thus, constituent elements having the same functions as the functions in the first embodiment are denoted by the same reference characters as the reference characters in the first embodiment, and description will be omitted.

### <Modification 1 of First Embodiment>

A distal end member 10A of an endoscope 1A of the present modification shown in FIG. 9 has flow direction correction members 70 for controlling the fluid discharging direction, on a distal end side of the third flow channel 17c. In other words, four walls of the second image pickup unit hole 55B that configure the third flow channel 17c are each provided with the flow direction correction member 70. The flow direction correction member 70 has an inclined surface which is inclined at an acute angle θ70 with respect to the wall surface of the second image pickup unit hole 55B and increases in height from the wall surface in the traveling direction of the fluid. The flow direction correction member 70 may be configured by a projection of the wall surface.

Four third flow channels 17c discharge fluids flowing through the respective third flow channels 17c to the inside of the body toward an axis which is an extension of a central axis OA of the lens unit 20 to the distal end side by means of the respective flow direction correction members 70. Thus, four flows of the fluid discharged through the four third flow channels 17c collide within the body and are stirred. As s a result, on the side far from the point of collision, the fluids discharged through the third flow channels 17c have a smaller flow velocity distribution than the flow near the tip member 10.

The endoscope 1A can reduce scattering and movement of a target as compared with the endoscope 1 even if a water supply amount is increased.

### <Modification 2 of First Embodiment>

A distal end member 10B of an endoscope 1B of Modification 2 shown in FIG. 10 has a flow direction correction member 71 on the wall 61a which is a flow buffer wall. The flow direction correction member 71 has a wall surface inclined at an acute angle with respect to the wall 61a. The flow direction correction member 71 may be a projection of the wall 61a.

In the second flow channel 17b in which the flow direction is considerably bent by the wall 61a, a region in which the flow velocity is locally fast occurs. The distal end member 10B can reduce the flow velocity distribution in the third flow channels 17c because the flow direction correction member 71 guides the fluid along the inclined surface.

### <Modification 3 to Modification 6 of First Embodiment>

A distal end member 10C of an endoscope 1C of Modification 3 shown in FIG. 11 has a recess 67A in the wall 61a. The fluid having flown in the recess 67A is stirred inside, and collides with the fluid from the first flow channel 17a. The cross-sectional shape of the recess 67A may be circular or polygonal. As will be described later, the distal end member 10C may have a plurality of recesses 67A.

For example, a distal end member 10D of an endoscope 1D of Modification 4 shown in FIG. 12 has a plurality of slit-like recesses 67B in the wall 61a.

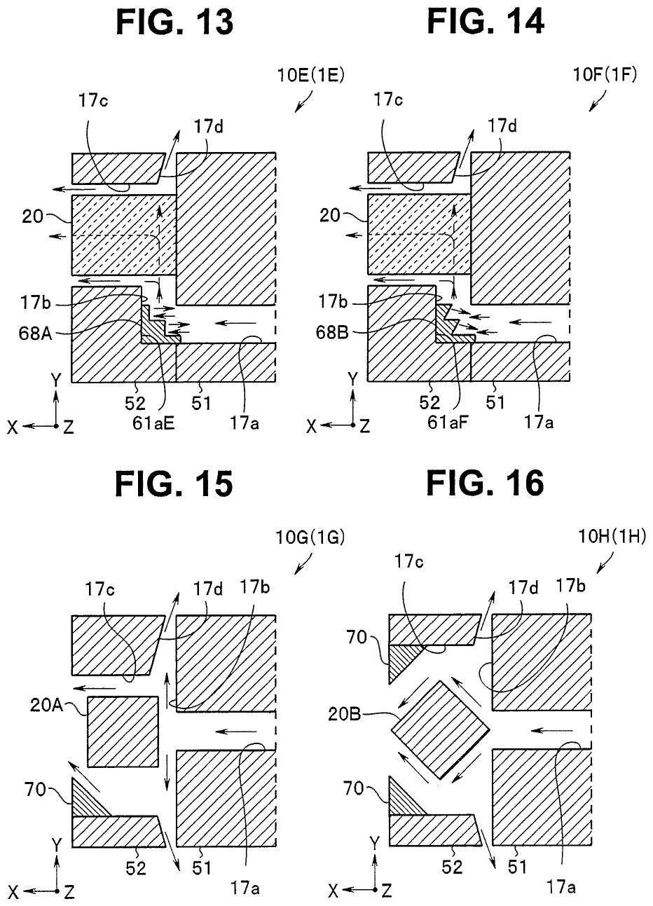

In a distal end member 10E of an endoscope 1E of Modification 5 shown in FIG. 13, a wall 61aE is not a plane but has a plurality of steps. The fluid having collided with a stepped surface is reversed in flow direction, so that the flow velocity distribution is improved. The stepped surfaces may be configured by a plurality of plates (flow branching and reversing members) protruding from the wall surface, and not only reversing the flow direction but also dividing the flow. The stepped surfaces may be inclined.

For example, a distal end member 10F of an endoscope 1F of Modification 6 shown in FIG. 14 has a plurality of steps on a wall 61aF, each of the steps having an inclined surface.

### <Modification 7 of First Embodiment>

In a distal end member 10G of an endoscope 1G of Modification 7 shown in FIG. 15, the fluid having passed through the first flow channel 17a collides with a lens unit 20A arranged in the second flow channel 17b and are divided. The lens unit 20A has functions of a flow buffer wall and a flow dividing member. By commonalizing the plurality of functional members, the distal end member 10G is compact, and the insertion section 2 of the endoscope 1G is small-diameter.

Note that in the distal end member 10G, the flow direction correction member 70 is disposed on at least any of four wall surfaces of the third flow channel 17c. In other words, the third flow channel 17c has a wall surface on which the flow direction correction member 70 is not disposed. An opening at which the flow direction correction member 70 is not disposed is not reduced in opening area by the flow direction correction member 70, so that the flow velocity of the fluid is not accelerated. The effect exerted by collision of the discharged fluid can be obtained if the flow direction correction member 70 is disposed on at least any of the walls.

### <Modification 8 of First Embodiment>

In a distal end member 10H of an endoscope 1H of Modification 8 shown in FIG. 16, the fluid having passed through the first flow channel 17a collides with a prism 20B which is a member separate from the lens unit arranged in the second flow channel 17b, and is divided. The prism 20B has the functions of a flow buffer wall and a flow dividing member. A plurality of flows as divided flow from the distal end surface 10SA in directions that the flows collide with each other along side surfaces of the prism 20B. A member having the functions of a flow buffer wall and a flow dividing member may be a cylinder or a polygonal prism instead of the prism 20B.

### <Modifications 9 to 12 of First Embodiment>

### <Modification 9 of First Embodiment>

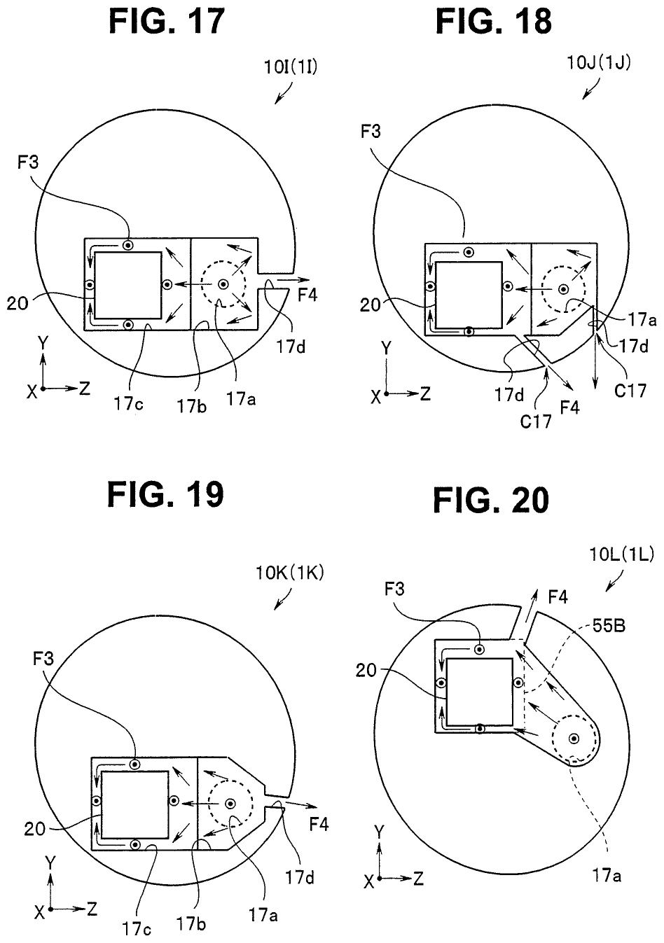

In a distal end member 10I of an endoscope 1I of Modification 9 shown in FIG. 17, the width (the Y-axis dimension) of the second flow channel 17b is the same. In other words, a guide groove of the second distal end member 52 has a rectangular opening shape.

### <Modification 10 of First Embodiment>

A guide groove of a distal end member 10J of an endoscope 1J of Modification 10 shown in FIG. 18 is a rectangle having a corner chamfered in conformity with the shape of an outer circumferential surface of the distal end member 10J. The distal end member 10J not only reduces a dead space in the flow channels, but also enables the second flow channel 17b to be efficiently arranged in the small-diameter distal end member 10J.

Note that the distal end member 10J has two cutouts C17. In other words, the distal end member of any embodiment or any modification may have a plurality of cutouts C17 (the fourth flow channels 17d) as already described.

As shown in FIG. 18, cross-sectional areas of the plurality of fourth flow channels 17d (opening areas of the plurality of cutouts C17) may vary. Further, the plurality of fourth flow channels 17d may be configured such that fluids F4 discharged from the respective fourth flow channels 17d collide.

### <Modification 11 of First Embodiment>

A guide groove of a distal end member 10K of an endoscope 1K of Modification 11 shown in FIG. 19 not only has a corner chamfered in conformity with the shape of an outer circumferential surface of the distal end member 10K, but also has the other corner chamfered so as to become symmetric. Thus, a region in which the flow velocity becomes locally fast does not occur in the fluid flowing in the second flow channel 17b.

### <Modification 12 of First Embodiment>

The lens unit 20 of a distal end member 10L of an endoscope 1L of Modification 12 shown in FIG. 20 is arranged at a position off-centered with respect to the image pickup unit hole 55B. In other words, the width of the third flow channel positioned on the upper side in the Y-axis direction is larger than the width of the third flow channel positioned on the lower side.

The third flow channel positioned on the upper side might have a flow quantity smaller than the flow quantity of the third flow channel positioned on the lower side close to the first flow channel 17a because the third flow channel positioned on the upper side is distant from the first flow channel 17a. However, the distal end member 10L equalizes the flow quantity in the four third flow channels by off-centering the arrangement position of the lens unit 20.

### Second Embodiment

A second embodiment is similar to the first embodiment, and has the same effects as the effects of the first embodiment. Thus, constituent elements having the same functions as the functions in the first embodiment are denoted by the same reference characters as the reference characters in the first embodiment, and description will be omitted.

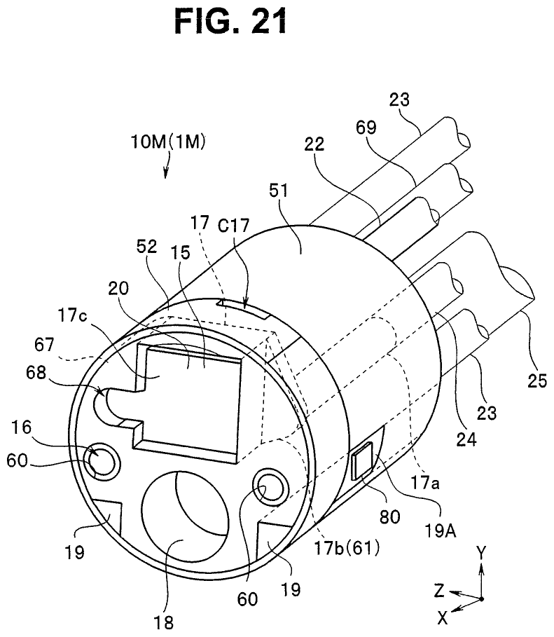

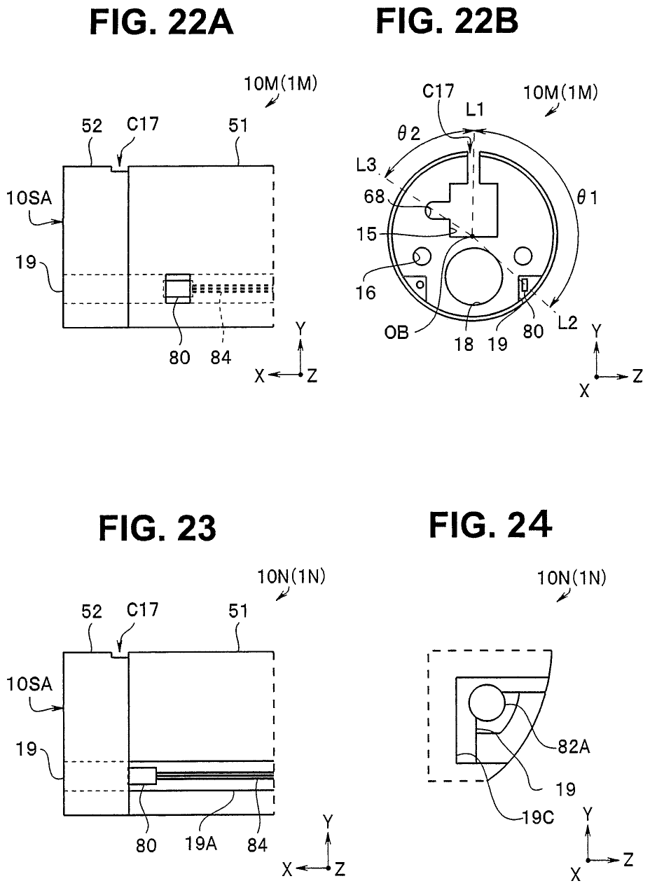

A distal end member 10M of an endoscope 1M of the present embodiment shown in FIG. 21, FIG. 22A, and FIG. 22B is provided with a probe insertion hole 68 communicating with the third flow channel 17c. The distal end member 10M has two illumination unit holes 60. A lithotriptic laser probe (not shown) is inserted in the probe insertion hole 68 when in use. Illumination units 16 are inserted in the two illumination unit holes 60, respectively.

In the distal end member 10M, at least one sensor 80 that measures a physical quantity is disposed in two sensor holes 19 respectively having openings in the distal end surface 10SA and the outer circumferential surface 10SS. The sensor 80 is, for example, a thermistor that detects the temperature, a thermocouple, or a pressure sensor that detects the pressure. The two sensor holes 19 may communicate with each other via a water channel in the distal end member 10M.

As shown in FIG. 22B, a first angle θ1 formed by a first line L1 that connects a central axis OB of the distal end member 10M and a second opening of the cutout C17 and a second line L2 that connects the central axis and the sensor 80 should only be more than 10 degrees, for example. Note that it is particularly preferable that the first angle θ1 should be more than 30 degrees.

A second angle θ2 formed by the first line L1 and a third line L3 that connects the probe insertion hole 68 and the central axis OB should also only be more than 10 degrees, for example. Note that it is particularly preferable that second angle θ2 should be more than 30 degrees.

If the first angle θ1 is more than 10 degrees, the sensor 80 is less likely to be affected by the fluid discharged through the cutout C17. If the second angle θ2 is more than 10 degrees, the laser probe is less likely to be affected by the fluid discharged through the cutout C17.

If the first angle θ1 is more than 45 degrees, erroneous detection of the temperature to be low and erroneous detection of the pressure to be high due to a cooling effect caused by the fluid discharged through the cutout C17 hitting the sensor 80 are prevented.

The sensor 80 is disposed in the cutout in the outer circumferential surface 10SS of the first distal end member 51. The cutout (an opening 19A) in the outer circumferential surface of the first distal end member 51 communicates with the sensor hole 19 having an opening in the second distal end member 52. The sensor 80 may be disposed in the opening 19A in the outer circumferential surface 10SS of the sensor hole 19 that allows insertion of the first distal end member 51 and is provided to extend to the second distal end member 52.

In the sensor hole 19 having a plurality of openings, the fluid passes through the sensor hole 19, so that the sensor 80 can correctly detect a physical quantity of the fluid around the distal end member 10M. Even if any of the openings is blocked by a body tissue, fragmented calculi, or the like, the fluid flows in through another one of the openings, which enables the sensor 80 to stably detect the physical quantity of the fluid. The shape of the cross section (the YZ plane) of the sensor hole 19 orthogonal to a long axis (the X-axis) and the opening shape in the distal end surface 10SA may be circular or substantially triangular in conformity with the outer diameter of the distal end member 10M.

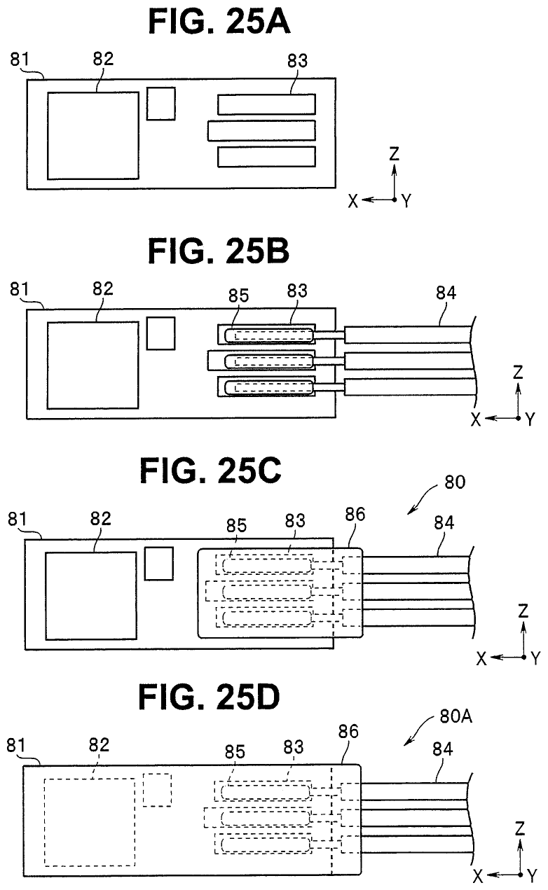

As shown in FIG. 22A, part of a detection unit 82 (see FIG. 25A) of the sensor 80 and wires 84 are not exposed to the opening 19A in the outer circumferential surface of the sensor hole 19. In other words, the detection unit 82 and the wires 84 are protected by the inner surface of the sensor hole 19. Note that as in a distal end member 10N of an endoscope 1N shown in FIG. 23, only part of the detection unit 82 may be protected.

However, when the detection unit of the sensor 80 comes into contact with the inner surface of the sensor hole 19, the detection accuracy might be degraded. Thus, as shown in FIG. 24, an inner surface 19C of the sensor hole 19 in which a substantially spherical detection unit 82A is arranged, for example, may be counterbored to become a space wider than the surroundings.

Instead of counterboring, the detection accuracy of the sensor may be secured by disposing a projecting member on the inner surface of the sensor hole 19 and arranging the wires of the sensor 80 on the projecting member.

In the endoscope 1M shown in FIG. 22B, the two sensors 80 that detect different physical quantities are arranged in the respective sensor holes 19. However, a plurality of sensors may be integrated. The plurality of sensors include a pressure sensor 81 and a temperature sensor 81A, for example.

The pressure sensor 81 shown in FIG. 25A is a flat plate having the detection unit 82 and an electrode 83 on a front surface. As shown in FIG. 25B, the wires 84 are bonded to the electrode 83 using a solder 85, for example. As shown in FIG. 25C, it is preferable that a sealing resin 86 made of epoxy resin, for example, should be disposed for sealing a bonding portion and fixing the arrangement position of the plurality of wires 84.

In a case where the detection unit 82 might be shorted due to a sensor configuration, the surface of the detection unit 82 may also be covered by the sealing resin 86 as shown in FIG. 25D.

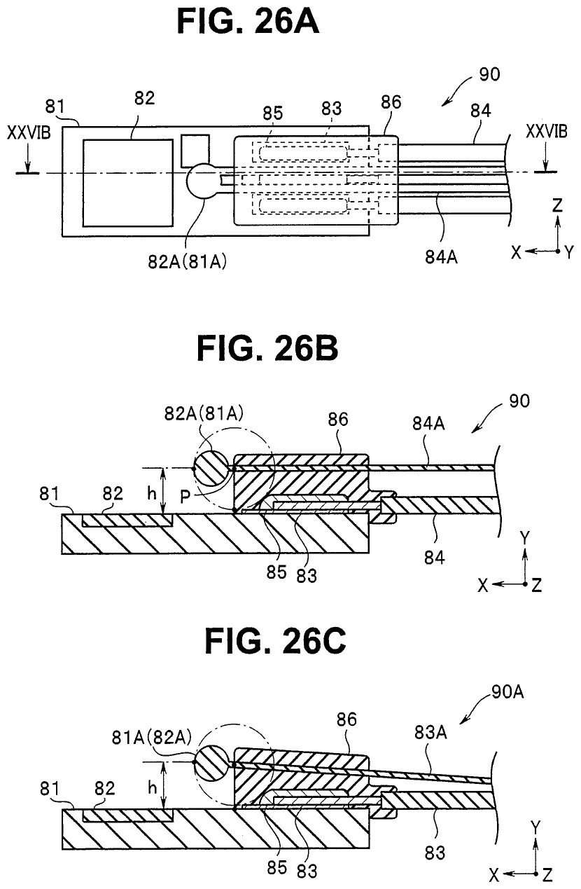

A composite sensor 90 in which the pressure sensor 81 and the temperature sensor 81A are integrated is shown in FIG. 26A and FIG. 26B. The temperature sensor 81A is a thermistor or a thermocouple having the substantially spherical detection unit 82A and wires 84A. The temperature sensor 81A is fixed to a detection surface of the pressure sensor 81 by the sealing resin 86. The two sensors can easily be integrated by applying the sealing resin to the pressure sensor 81, arranging the temperature sensor 81A, and then performing curing treatment. The composite sensor 90 can be arranged in a small space of the distal end member.

Note that the wires 84 of the pressure sensor 81 and the wires 84A of the temperature sensor 81A may be attached or twisted to improve handling of the composite sensor 90.

In order to prevent the accuracy and responsiveness of each of the temperature sensor 81A and the pressure sensor 81 from being adversely affected, it is preferable that the detection unit 82A of the temperature sensor 81A should be exposed without being covered by the sealing resin 86 and the detection unit 82A should not be in touch with the detection unit 82 of the pressure sensor 81.

In the composite sensor 90, the detection unit 82A of the temperature sensor 81A is arranged between the detection unit 82 and the electrode 83 of the pressure sensor 81. As shown in FIG. 26B, a base point height h of a base point P of the detection unit 82A of the temperature sensor 81A from the surface of the pressure sensor 81 is set such that an imaginary circle C centering on the base point P and including a distal end of the detection unit 82A in an arc does not come into contact with the pressure sensor 81.

In a composite sensor 90A shown in FIG. 26C, the temperature sensor 81A is inclined with respect to the surface of the pressure sensor 81 in order to secure the base point height h. If a sufficient base point height h can be ensured, the detection unit 82A may be arranged on the detection unit 82.

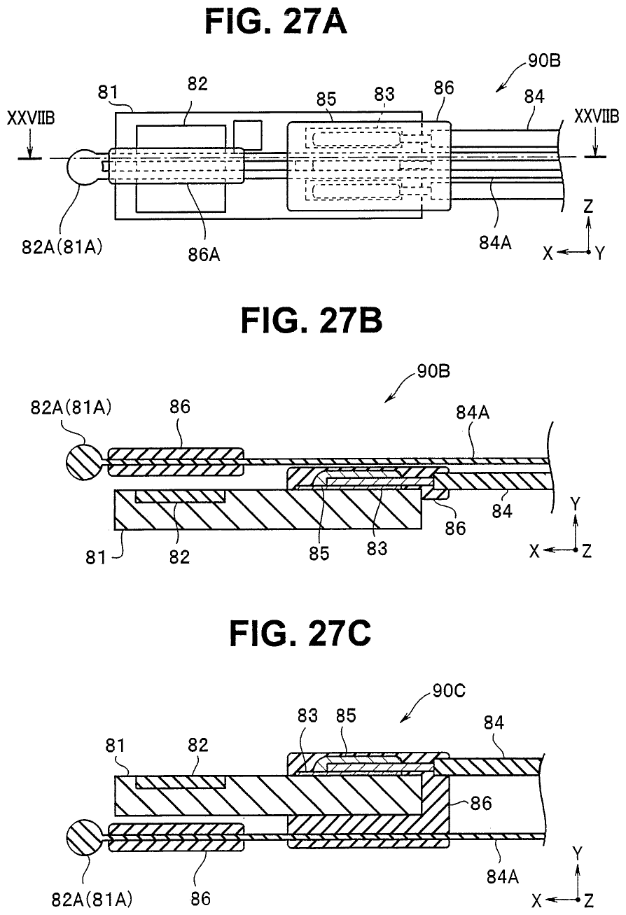

In a composite sensor 90B shown in FIG. 27A and FIG. 27B, the detection unit 82A of the temperature sensor 81A protrudes from a distal end of the pressure sensor 81. The composite sensor 90B can be laid out in a space-saving manner because of a small thickness as compared with the composite sensor 90 or the like. Note that in a case where the whole surface of the pressure sensor 81 is sealed, the detection unit 82A of the temperature sensor 81A may protrude from the distal end of the pressure sensor 81 similarly to the composite sensor 90B.

In a composite sensor 90C shown in FIG. 27C, the temperature sensor 81A is arranged on a rear surface opposite to the front surface on which the detection unit 82 of the pressure sensor 81 is arranged. The detection unit 82 protrudes from the distal end of the pressure sensor 81. The sealing resin 86 may be applied to the whole region of the rear surface of the composite sensor 90C.

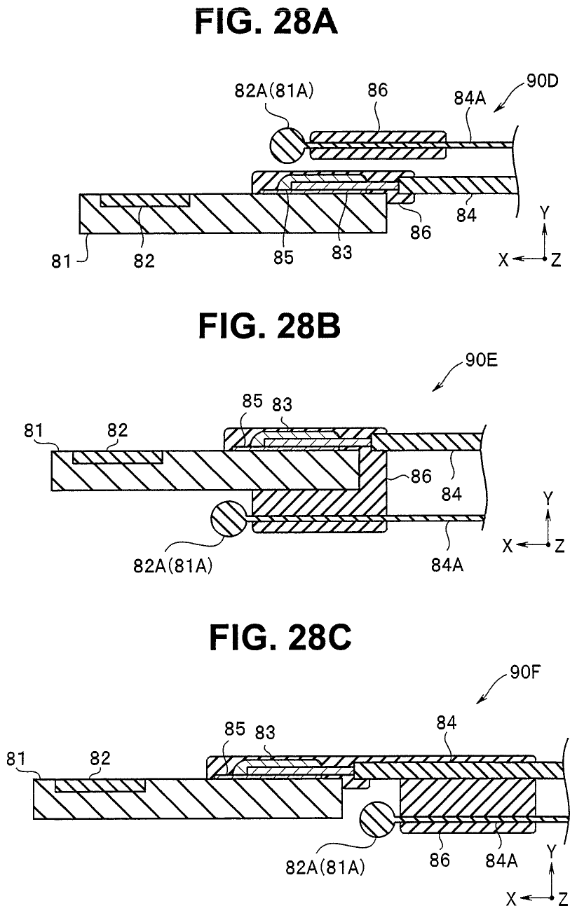

In a composite sensor 90D shown in FIG. 28A, the detection unit 82A of the temperature sensor 81A is arranged on the electrode 83 of the pressure sensor 81.

In a composite sensor 90E shown in FIG. 28B, the temperature sensor 81A is arranged on the rear surface of the pressure sensor 81. The sealing resin 86 seals the pressure sensor 81 and the temperature sensor 81A.

In a composite sensor 90F shown in FIG. 28C, the detection unit 82A of the temperature sensor 81A is arranged backward on the rear surface of the pressure sensor 81. The sealing resin 86 seals the pressure sensor 81 and the temperature sensor 81A.

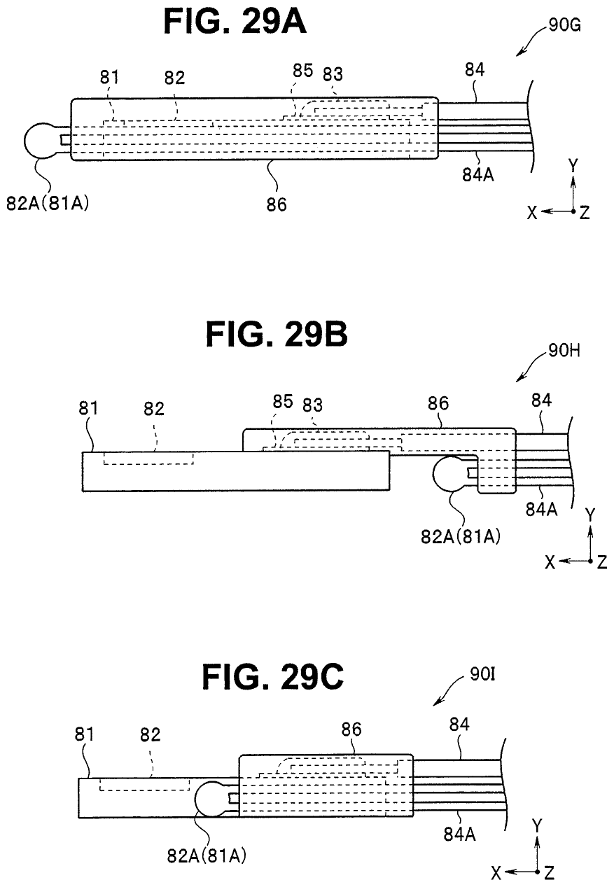

In a composite sensor 90G shown in FIG. 29A, the detection unit 82A of the temperature sensor 81A is arranged forward on a side surface of the pressure sensor 81. In other words, the wires 84A of the temperature sensor 81A are arranged on the side surface side of the pressure sensor 81 (in the Z-axis direction). The sealing resin 86 seals the pressure sensor 81 and the temperature sensor 81A.

In a composite sensor 90H shown in FIG. 29B, the detection unit 82A of the temperature sensor 81A is arranged backward on a side surface of the pressure sensor 81. In other words, in the drawing, the temperature sensor 81A is arranged on the near side on the sheet of drawing relative to the side surface of the pressure sensor 81.

In a composite sensor 90I shown in FIG. 29C, the detection unit 82A of the temperature sensor 81A is arranged substantially at the center of a side surface of the pressure sensor 81. In other words, the wires 84A of the temperature sensor 81A are arranged on the side surface side of the pressure sensor 81 (the near side on the sheet of drawing).

As described above, each of the composite sensors 90A to 901 has favorable detection accuracy and responsiveness although the arrangement position of the temperature sensor 81A on the pressure sensor 81 varies.

### <Modifications of Second Embodiment>

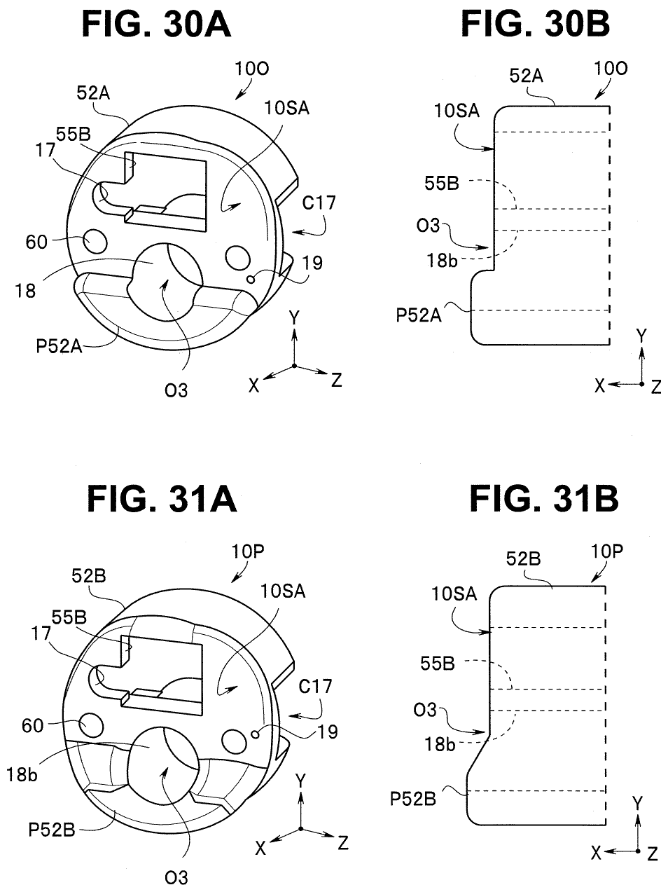

Modifications 1 to 4 of the second embodiment will be described with reference to FIG. 30A to FIG. 33B. Modifications 1 to 4 of the second embodiment are similar to the second embodiment. Thus, constituent elements having the same functions as the functions in the second embodiment are denoted by the same reference characters as the reference characters in the second embodiment, and description will be omitted. Note that hereinafter, projections protruding forward with respect to the distal end surface 10SA of the second distal end member 52 in the already-described drawings (for example, FIG. 5) will be referred to as projecting regions P52A to P52D.

If a mucosal tissue is suctioned and the third opening O3 of the treatment instrument hole 18 is blocked when a fluid is suctioned through the treatment instrument hole 18 of the distal end member 10 of the endoscope 1 inserted into the body, not only suction can no longer be performed, but also the mucosal tissue might be damaged.

A distal end member 10O of Modification 1 of the second embodiment shown in FIG. 30A and FIG. 30B has the projecting region P52A protruding from the distal end surface 10SA on the lower side of the outer circumference of the third opening O3. Thus, a difference in level formed by the projecting region P52A creates a gap, and thus prevents the third opening O3 from being completely blocked even if a mucosal tissue is suctioned by the treatment instrument hole 18.

For example, the projecting region P52A is a projection that surrounds a half or more of the circumference of the third opening O3 having a diameter of 1 mm, has a planar top, and has a height of 0.4 mm.

Note that it is preferable that the projecting region P52A should be arranged in the neighborhood of the outer circumference of the distal end surface 10SA in order to avoid suction at a place adjacent to a mucosal tissue.

A distal end member 10P of Modification 2 of the second embodiment shown in FIG. 31A and FIG. 31B has the projecting region P52B having a planar top and an inclined surface on the lower side of the circumference of the third opening O3 (the outer circumferential side). In other words, the projecting region P52B has an inclined surface inclined from the top plane toward the distal end surface 10SA.

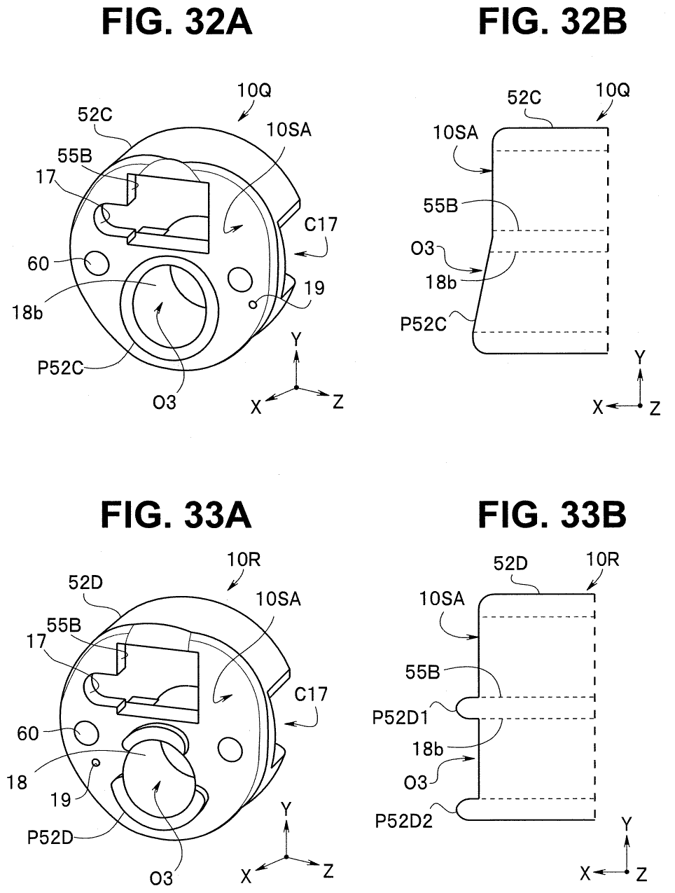

A distal end member 10Q of Modification 3 of the second embodiment shown in FIG. 32A and FIG. 32B has the projecting region P52C formed of an inclined surface on the lower side of the circumference of the third opening O3.

A distal end member 10R of Modification 4 of the second embodiment shown in FIG. 33A and FIG. 33B has a first projecting region P52D1 and a second projecting region P52D2. The first projecting region P52D1 is arranged on the upper side (the inner circumferential side) of the circumference of the third opening O3 in the drawing. The second projecting region P52D2 is arranged on the lower side (the outer circumferential side) of the circumference of the third opening O3.

The projecting regions may have a planar top, and may have a plurality of protrusions. The projecting regions may have a shape in which a plurality of protrusions connect to each other.

Note that the present invention is not limited to the above-described respective embodiments, and can be variously modified or changed. They also fall within the technical scope of the present invention.

In the above-described respective embodiments, the configurations in which the whole circumference of the lens unit 20 is arranged inside the third flow channel 17c have been described. However, the present invention is not limited to such configurations. For example, a configuration in which part of the outer circumferential portion of the lens unit 20 is arranged inside the third flow channel 17c can be adopted. It goes without saying that the configurations of the above-described respective embodiments may be combined as appropriate.

What is claimed is:

1. Insertion equipment comprising:
an insertion section inserted into a subject;
a distal end member provided at a distal end of the insertion section; and
a tube configured to supply a fluid to the distal end member, wherein
in the distal end member, the fluid passes through a first flow channel extending in a distal end direction, passes through a second flow channel in a direction different from the distal end direction, is divided, is discharged through a first opening in a distal end surface, and part of the fluid is discharged through one or more second openings in an outer circumferential surface of the distal end member in a radial direction or a proximal end direction.

2. The insertion equipment according to claim 1, wherein
the distal end member has an image pickup unit having a substantially prismatic shape, and
the fluid is divided by the image pickup unit which is a flow dividing member.

3. The insertion equipment according to claim 2, wherein the fluid is discharged through the first opening having a frame shape in the distal end direction along at least one side surface of the image pickup unit.

4. The insertion equipment according to claim 3, wherein
the distal end member has a flow direction correction member, and
at least part of the fluid discharged from the distal end surface is discharged in a direction that crosses a central axis of the image pickup unit by the flow direction correction member.

5. The insertion equipment according to claim 1, wherein the distal end member has a wall with which the fluid having passed through the first flow channel collides, and the fluid flows in a direction different from the distal end direction along the wall.

6. The insertion equipment according to claim 1, wherein a value obtained by adding an area of the first opening and an area of the second opening is larger than a cross-sectional area of the first flow channel.

7. The insertion equipment according to claim 1, wherein the insertion equipment is an endoscope used in an endoscope system configured to perfuse a liquid between an inside and an outside of the subject.

8. Insertion equipment comprising:
an insertion section inserted into a subject;
a distal end member provided at a distal end of the insertion section;
at least one sensor disposed in the distal end member and configured to detect a physical quantity; and
a tube configured to supply a fluid to the distal end member, wherein
in the distal end member, the fluid passes through a first flow channel extending in a distal end direction, passes through a second flow channel in a direction different from the distal end direction, is divided, is discharged through a first opening in a distal end surface, and part of the fluid passes through the second flow channel and is discharged through one or more second openings in an outer circumferential surface of the distal end member, and
a first angle formed by a first line that connects a central axis of the distal end member and the second openings and a second line that connects the central axis and the sensor is more than 10 degrees.

9. The insertion equipment according to claim 8, wherein
the distal end member has a probe insertion hole in which a laser probe is inserted, and
a second angle formed by the first line and a third line that connects the central axis and an opening of the probe insertion hole is more than 10 degrees.

10. The insertion equipment according to claim 8, wherein the sensor is disposed in a sensor hole of the distal end member having openings in the distal end surface and the outer circumferential surface, respectively.

11. The insertion equipment according to claim 9, wherein the fluid is discharged through the second openings in a proximal end direction.

12. The insertion equipment according to claim 11, comprising a third opening configured to suction the fluid in the distal end surface.

13. The insertion equipment according to claim 12, comprising at least one projecting region around the third opening, the projecting region protruding from the distal end surface.

14. The insertion equipment according to claim 13, wherein a distal end of the projecting region is a plane, and the projecting region has an inclined surface inclined from the plane at the distal end toward the distal end surface of the distal end member.

15. The insertion equipment according to claim 14, wherein
the distal end member has an image pickup unit, and
the fluid is divided by the image pickup unit which is a flow dividing member.

16. The insertion equipment according to claim 15, wherein the image pickup unit is substantially prismatic, and the fluid is discharged through the first opening having a frame shape in the distal end direction along one or more side surfaces of the image pickup unit.

17. The insertion equipment according to claim 16, wherein the distal end member has a first distal end member and a second distal end member arranged on a distal end side of the first distal end member.

18. The insertion equipment according to claim 17, wherein the second distal end member configures the first opening in conjunction with the image pickup unit.

19. The insertion equipment according to claim 18, wherein the second distal end member has a cutout that serves as the second openings in an outer circumferential surface.

20. The insertion equipment according to claim 19, wherein the cutout in the second distal end member forms the second flow channel by arranging the second distal end member in the first distal end member.
